(19) United States
(12) Patent Application Publication
(10) Pub. No.: US 2010/0030097 A1
(43) Pub. Date: Feb. 4, 2010

(54) METHOD TO DETERMINE THE ATTRIBUTES ASSOCIATED WITH A BRAND OR PRODUCT

(76) Inventor: Richard Bernard Silberstein, Victoria (AU)

Correspondence Address: MASTERMIND IP LAW PC, 421-A SANTA MARINA COURT, ESCONDIDO, CA, 92029, US

(21) Appl. No.: 12/520,868

(22) PCT Filed: Dec. 22, 2006
(86) PCT No.: PCT/AU2006/002007

§ 371 Date: Jun. 22, 2009

(51) Int. Cl. A61B 5/0484

(52) U.S. Cl. 600/544

(57) ABSTRACT

A method of evaluating characteristics of a brand or product including the steps of: (a) presenting the brand or product to the subject during a first period; (b) determining brain activity of the subject during the first period; (c) presenting neutral visual and/or audio material to a subject during a second period; (d) determining a reference level of brain activity of the subject during the second period; and (e) evaluating attributes associated by the subject with the brand or product by determining differences in brain activities between the first and second periods.

The qualities associated in the mind of the consumer with a brand or product are a matter of profound commercial significance. The manner in which the brand or product is perceived by the consumer has a powerful impact on the likely purchase behaviour of the consumer. The value attributed to the brand is increasingly an important component of the value of a modern corporation. The value of a brand is fundamentally determined by the attitude of the consumer to that brand. Feelings of trust and loyalty, for example will be associated with high brand value. Brand managers also seek to associate specific qualities with a brand, for example, car safety and a particular brand of car or innovation and a certain high-technology manufacturer.

At present, such associations are determined by questionnaires, focus group discussion and in-depth interviews. It is widely recognized that these techniques are deficient in that they rely on the verbal responses of the subjects. Such verbal responses are considered a poor indicator of the emotional qualities associated with the brand.

This invention discloses a method that relies on the measurement of brain activity, rather than verbal responses to determining the various psychological qualities associated with any brand or product.

A first aspect of the invention is concerned with assessment of general characteristics associated with a brand or product.

According to a first aspect of the invention there is provided a method of evaluating characteristics of a brand or product including the steps of:

(a) presenting the brand or product to the subject during a first period;

(b) determining brain activity of the subject during the first period;

(c) presenting neutral visual and/or audio material to a subject during a second period;

(d) determining a reference level of brain activity of the subject during the second period; and

(e) evaluating attributes associated by the subject with the brand or product by determining differences in brain activities between said first and second periods.

A second aspect of the invention is concerned with attributes specifically associated with a brand or product.

According to a second aspect of the invention there is provided a method of determining attributes associated with a brand or product including the steps of:

(a) simultaneously presenting the brand or product and a semantic probe to the subject during a first period;

(b) determining brain activity of the subject during the first period;

(c) presenting the neutral visual and/or audio material to a subject during a second period;

(d) determining a reference level of brain activity of the subject during said second period; and

(e) determining whether there is congruence or incongruence between the attributes associated with the brand or product and the semantic probe by assessing whether there is an increase or decrease in brain activity in step (b) compared to step (d).

The invention also provides a system for determining attributes associated with a brand or product including:

display means for displaying the brand or product image to a subject;

brain activity determining means for determining brain activity of the subject; and

assessment means coupled to receive first output signals from said brain activity determining means in a first period in which the brand or product image is displayed to the subject and to receive second output originals from said brain activity determining means in a second period in which neutral material is displayed to the subject in order to establish a reference level of brain activity, the assessment means being operable to assess differences between said first and second output signals.

The invention also provides a system for determining attributes associated with a brand or product including:

display means for displaying the brand or product image to a subject;

brain activity determining means for determining brain activity of the subject; and

assessment means coupled to receive first output signals from said brain activity determining means in a first period in which the brand or product image is displayed to the subject simultaneously with a semantic probe and to receive second output originals from said brain activity determining means in a second period in which neutral material is displayed to the subject in order to establish a reference level of brain activity, the assessment means being operable to assess differences between said first and second output signals.

Preferably, brain activity is measured while subjects view a brand image or a product image, represented by the brand name and logo on a video display or a visual presentation of the product and product name. The brand or product image remains on the screen for an initial duration of 0.5 sec to 5 sec, preferably, 1 sec. During a subsequent period, the brand or product image remains on the screen and a word describing a quality or semantic probe appears under the brand or product image. The duration of the subsequent period is 0.5 sec to 5 sec, preferably the same duration as the initial period.

Brand image and product image need not be static and a moving product image or brand image may be used also. The semantic probe is most commonly one or more words but may also be a sound or another image.

### Evaluating General Brand or Product Characteristics

The psychological response to the brand or product image alone can be ascertained from the distribution of brain activity during the Initial period when the brand or product image is presented.

### Visual Attention to Detail

This measure refers to the level of visual attention to detail or text elicited by the brand or product image during the initial period.

SSVEP phase advance or amplitude change at the left occipital region, preferably electrode O₁ in the International 10-20 system has been found to be relevant to assessment of the subject's Visual Attention to Detail. If inverse mapping techniques are used, the relevant location in the left cerebral cortex is the vicinity of Brodmans area 17.

### Visual Attention to Global Features

This measure refers to the level of visual attention to global features elicited by the brand or product image during the initial period.

SSVEP phase advance or amplitude change at the right occipital region, preferably electrode O₂ in the International 10-20 system has been found to be relevant to the assessment of the subject's Visual Attention to Global Features including responses to facial expressions displayed on the screen. If inverse mapping techniques are used, the relevant location in the right cerebral cortex is the vicinity of Brodmans area 17.

The desirability associated with the brand or product image is indicated by increased brain activity at left and right parietal recording sites during the initial period. In the International 10-20 system that labels recording sites on the brain, the positions referred to above correspond to the vicinity of P₃ and P₄. If inverse mapping techniques are used, the relevant location in the left cerebral cortex is the vicinity of the left and intraparietal areas.

The Emotional intensity associated with the brand or product image is indicated by increased brain activity at right parieto-temporal region, preferable approximately equidistant from right hemisphere electrodes O₂, P₄ and T₆ during the initial period. If inverse mapping techniques are used, the relevant location in the right cerebral cortex is the vicinity of the right parieto-temporal junction.

How well the brand image or product image is stored or encoded in long-term memory is indicated by increased brain activity at left and right temporal sites in the vicinity of T₅ and T₆ and also at right frontal sites equidistant between C₄, F₄ and F₈ during the initial period. If inverse mapping techniques are used, the relevant locations in the left and right temporal lobes in the vicinity of Brodman's area 20 and in the right frontal cortex in the vicinity of Brodmans areas 6, 44, 45, 46 and 47.

The extent to which individuals are attracted or repelled by the brand image or product image is given by the difference between brain activity at left frontal/prefrontal and right frontal/prefrontal regions. Attraction is indicated by a larger activity in the left hemisphere compared to the right while repulsion is indicated by greater activity in the right hemisphere compared to the left.

Attraction=(a₁*brain activity recorded at electrode F₃+a₂*brain activity recorded at electrode Fₚ₁−a₃*brain activity recorded at electrode F₄−a₄*brain activity recorded at electrode Fₚ₂) Equation 1

where a=a₂=a₃=a₄=1.0

A positive value for the attraction measure is associated with the participants finding the material attractive and liked while a negative measure is associated with repulsion or dislike.

If inverse mapping techniques are used, the relevant expression is:

Attraction=(c₁*brain activity at right orbito-brain activity at frontal cortex (in vicinity of Brodman area 11)+c₂*brain activity at right dorso-lateral prefrontal cortex (in vicinity of Brodman area 9)+c₃*brain activity at left orbito-frontal cortex (in vicinity of Brodman area 11)+c₄*brain activity at left dorso-lateral prefrontal cortex (vicinity of Brodman area 9)) Equation 2

where c₁=1, c₂=1, c₃₌₁, c₄₌₁

The extent to which the brand image or product image engages is given by the weighted mean brain activity during the initial period at prefrontal sites described by the expression below,

Engagement=(b₁*brain activity at electrode F₃+b₂*brain activity at electrode Pₚ₁+b₃*brain activity advance at electrode F₄+b₄*brain activity at Electrode Fₚ₂) Equation 3

where b₁=0.1, b₂=0.4, b₃=0.1, b₄=0.4

If inverse mapping techniques are used, the relevant expression is:

Engagement=(d₁*brain activity at right orbito-frontal cortex (in vicinity of Brodman area 11)+d₂*brain activity at right dorso-lateral prefrontal cortex (in vicinity of Brodman area 9)+d₃*brain activity at left orbito-frontal cortex (in vicinity of Brodman area 11)+d₄*brain activity at left dorso-lateral prefrontal cortex (in vicinity of Brodman area 9)) Equation 4

where: d₁=0.1, d₂=0.4, d₃=0.1, d₄=0.4

### Evaluating Specific Brand or Product Characteristics

The association between specific characteristics and the brand or product image is indicated by the distribution of brain activity during the appearance of the brand or product image and the semantic probe. Congruence between the semantic probe and the qualities associated with the brand or product image in the mind of the subjects is indicated by increased brain activity at right prefrontal sites, in the vicinity of electrode Fₚ₂. If inverse mapping techniques are used this corresponds to brain activity at right orbito-frontal cortex (in vicinity of Brodman area 11). Incongruence between the semantic probe and the qualities associated with the brand or product image in the mind of the subjects is indicated by reduced brain at right prefrontal sites, in the vicinity of electrode Fₚ₂. If inverse mapping techniques are used, these correspond to reduced brain activity at right orbito-brain activity at frontal cortex (in vicinity of Brodman area 11).

### Measuring Brain Activity

A number of methods are available for measuring brain activity. The main feature they must possess is adequate temporal resolution or the capacity to track the rapid changes in brain activity. Spontaneous brain electrical activity or the electroencephalogram (EEG) or the brain electrical activity evoked by a continuous visual flicker that is the Steady State Visually Evoked (SSVEP) are two examples of brain electrical activity that can be used to measure changes in brain activity with sufficient temporal resolution. The equivalent spontaneous magnetic brain activity or the magnetoencephalogram (MEG) and the brain magnetic activity evoked by a continuous visual flicker Steady State Visually Evoked Response (SSVER).

### Electroencephalogram and Magnetoencephalogram (EEG and MEG)

The EEG and MEG are the record of spontaneous brain electrical and magnetic activity recorded at or near the scalp surface. Brain activity can be assessed from the following EEG or MEG components.

### 1. Gamma or High Frequency EEG or MEG Activity

This is generally defined as EEG or MEG activity comprising frequencies between 35 Hz and 80 Hz. Increased levels of Gamma activity are associated with increased levels of brain activity, especially concerned with perception. (Fitzgibbon S P, Pope K J, Mackenzie L, Clark C R, Willoughby J O. Cognitive tasks augment gamma EEG power. Clin Neurophysiol. 2004:115:1802-1809).

If scalp EEG gamma activity is used as the indicator of brain activity, the relevant scalp recording sites are listed above. If EEG gamma activity at the specific brain regions listed above is used as the indicator brain activity then inverse mapping techniques such as LORETA must be used (Pascual-Marqui R, Michel C, Lehmann D (1994): Low resolution electromagnetic tomography: a new method for localizing electrical activity in the brain. Int J Psychophysiol 18:49-65).

If MEG gamma activity at the specific brain regions listed above is used as the indicator of brain activity, then the multi-detector MEG recording system must be used in conjunction with an MEG inverse mapping technique (see Uutela K, Ha\ddot{}ma\ddot{}la\ddot{}inen M, Somersalo E (1999): Visualization of magnetoencephalographic data using minimum current estimates. Neuroimage 10:173-180 and Fuchs M, Wagner M, Kohler T, Wischmann HA (1999): Linear and nonlinear current density reconstructions, J Clin Neurophysiol 16:267-295).

### 2. Frequency of EEG or MEG Alpha Activity

Brain activity may also be indexed by changes in the frequency of the ongoing EEG or MEG in the alpha frequency range (8.0 Hz-13.0 Hz). Increased frequency is an indication of increased activity. The frequency needs to me estimated with high temporal resolution. Two techniques that can be used to measure ‘instantaneous frequency’ are complex demodulation (Walter D, The Method of Complex Demodulation. Electroencephalog. Clin. Neurophysiol, 1968 Suppl 27:53-7) and the use of the Hilbert Transform (Leon Cohen, “Time-frequency analysis”, Prentice-Hall, 1995). Increased brain activity is indicated by an increase in the instantaneous frequency of the EEG in the alpha frequency range.

If the frequency of scalp EEG alpha activity is used as the indicator of brain activity, the relevant scalp recording sites are listed above. If the frequency of EEG alpha activity at the specific brain regions listed above is used as the indicator brain activity then inverse mapping techniques such as LORETA must be used (Pascual-Marqui R, Michel C, Lehmann D (1994): Low resolution electromagnetic tomography: a new method for localizing electrical activity in the brain. Int J Psychophysiol 18:49-65).

If the frequency of MEG alpha activity at the specific brain regions listed above is used as the indicator of brain activity, then the multi-detector MEG recording system must be used in conjunction with an MEG inverse mapping technique (see Uutela K, Ha\ddot{}ma\ddot{}la\ddot{}inen M, Somersalo E (1999): Visualization of magnetoencephalographic data using minimum current estimates. Neuroimage 10:173-180, and Fuchs M, Wagner M, Kohler T, Wischmann H A (1999): Linear and nonlinear current density reconstructions, J Clin Neurophysiol 16:267-295).

### 3. SSVEP or SSVER Phase as an Indicator of Brain Activity

Brain activity may also be indicated by the phase of the Steady State Visually Evoked Potential (SSVEP) or the Steady State Visually Evoked Response (SSVER).

U.S. Pat. Nos. 4,955,938, 5,331,969, and 6,792,304 (the contents of which are hereby incorporated herein by reference) disclose technique for obtaining a steady state visually evoked potential (SSVEP) from a subject. This technique can also be used to obtain a steady state visually evoked response (SSVER). These patents disclose the use of Fourier analysis in order to rapidly obtain the SSVEP and SSVER phase and changes thereto. The preferred way in which SSVEP and SSVER amplitudes and phases are calculated are summarised below.

### SSVEP and SSVER Amplitude and Phase

The digitized brain electrical activity (electroencephalogram or EEG) brain magnetic activity (MEG) together with timing of the stimulus zero crossings enables one to calculate the SSVEP or SSVER elicited by the flicker at a particular stimulus frequency from the recorded EEG or MEG or from EEG or MEG data that has been pre-processed using Independent Components Analysis (ICA) to remove artifacts and increase the signal to noise ratio. [Bell A. J and Sejnowski T. J. 1995, An information maximisation approach to blind separation and blind deconvolution, Neural Computation, 7, 6, 1129-1159; T-P. Jung, S. Makeig, M. Westerfield, J Townsend, E. Courchesne and T. J. Sejnowskik, Independent component analysis of single-trial event-related potential Human Brain Mapping, 14(3):168-85, 2001].

Calculation of SSVEP or SSVER amplitude and phase for each stimulus cycle for a given stimulus frequency. Calculation accomplished used Fourier techniques using Equations 5 and 6 below.

$${a}_{n} = \frac{1}{S Δτ} {\sum }_{i = 0}^{S - 1} f ( nT + i Δτ ) cos ( \frac{2 π}{T} ( nT + i Δτ ) ) \\ {b}_{n} = \frac{1}{S Δτ} {\sum }_{i = 0}^{S - 1} f ( nT + i Δτ ) sin ( \frac{2 π}{T} ( nT + i Δτ ) ) \qquad Equation 5$$

Calculation of SSVEP Fourier components where aₙ and bₙ are the cosine and sine Fourier coefficients respectively. n represents the nth stimulus cycle, S is the number of samples per stimulus cycle (16), Δτ is the time interval between samples, T is the period of one cycle and f(nT+iΔτc) is the EEG or MEG signal (raw or pre-processed using ICA).

$${SSVEP}_{amplitude} = \sqrt{( {A}_{n}^{2} + {B}_{n}^{2} )} or \\ {SSVER}_{amplitude} = \sqrt{( {A}_{n}^{2} + {B}_{n}^{2} )} \\ {SSVEP}_{phase} = a tan ( \frac{{B}_{n}}{{A}_{n}} ) or \\ {SSVER}_{phase} = a tan ( \frac{{B}_{n}}{{A}_{n}} ) \qquad Equation 6$$

Where Aₙ and Bₙ are overlapping smoothed Fourier coefficients calculated by using Equation 7 below.

$${A}_{n} = {\sum }_{i = 1}^{i = N} {a}_{n + i} / N \\ {B}_{n} = {\sum }_{i = 1}^{i = N} {b}_{n + i} / N \qquad Equation 7$$

Amplitude and phase components can be calculated using either single cycle Fourier coefficients (aₙ and bₙ) or coefficients that have been calculated by smoothing across multiple cycles (Aₙ and Bₙ).

Equations 6 and 7 describe the procedure for calculating the smoothed SSVEP or SSVER coefficients for a single subject. For pooled data, the SSVEP or SSVER coefficients (Aₙ and Bₙ) for a given electrode are averaged (or pooled) across all of the subjects or a selected group of subjects.

As the number of cycles used in the smoothing increases, the signal to noise ratio increases while the temporal resolution decreases. The number of cycles used in the smoothing is typically in excess of 5 and less than 130.

Equations 6 and 7 apply to scalp SSVEP data as well as brain electrical activity inferred at the cortical surface adjacent to the skull and deeper regions. Activity in deeper regions of the brain such as the orbito-frontal cortex or ventro-medial cortex can be determined using a number of available inverse mapping techniques such as EMSE (Source Signal Imaging, Inc, 2323 Broadway, Suite 102, San Diego, Calif. 92102, USA) and LORETA (Pascual-Marqui R, Michel C, Lehmann D (1994): Low resolution electromagnetic tomography: a new method for localizing electrical activity in the brain. Int J Psychophysiol 18:49-65). If the SSVER amplitude or phase changes at the specific brain regions listed above are used as the indicator of brain activity, then the multi-detector MEG recording system must be used in conjunction with an MEG inverse mapping technique (see Uutela K, Ha\ddot{}ma\ddot{}la\ddot{}inen M, Somersalo E (1999): Visualization of magnetoencephalographic data using minimum current estimates, Neuroimage 10:173-180, and Fuchs M, Wagner M; Kohler T. Wischmann HA (1999): Linear and nonlinear current density reconstructions, J Clin Neurophysiol 16:267-295).

The invention will now be further described with reference to the accompanying drawings, in which:

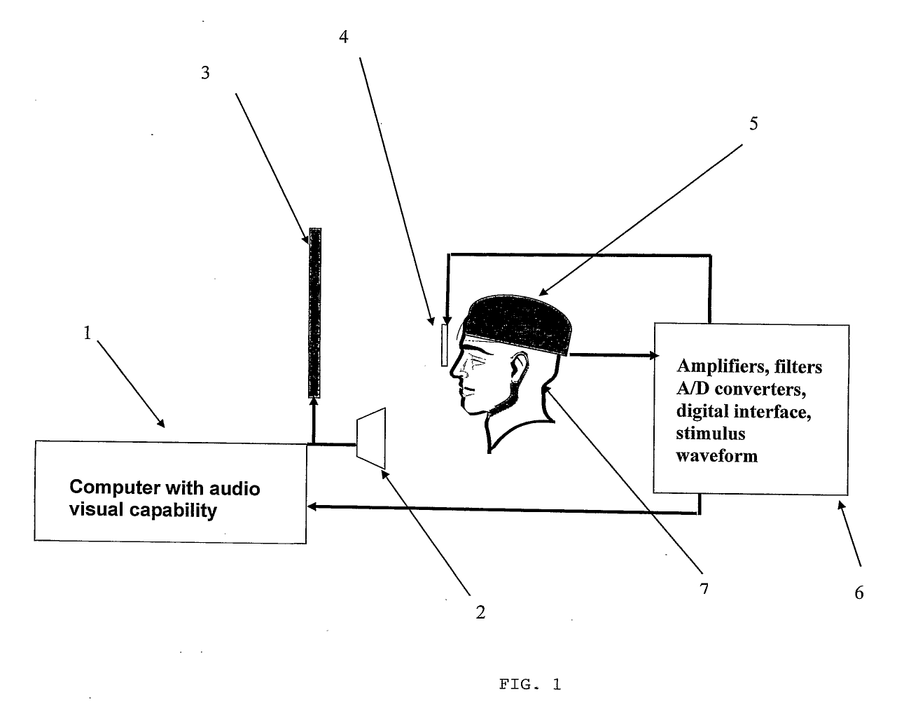

FIG. 1 is a schematic view of a system of the invention;

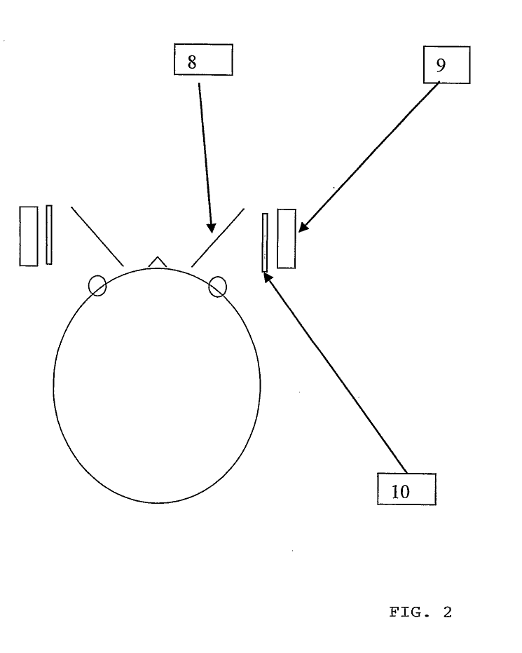

FIG. 2 is a schematic view showing in more detail the manner in which visual flicker stimuli are presented to a subject;

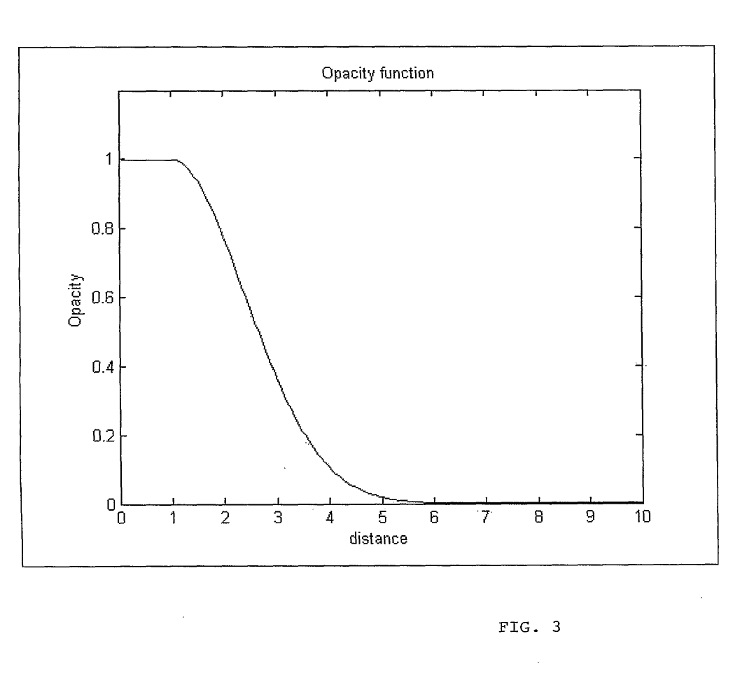

FIG. 3 is a graph showing the opacity of the screen as a function of radius;

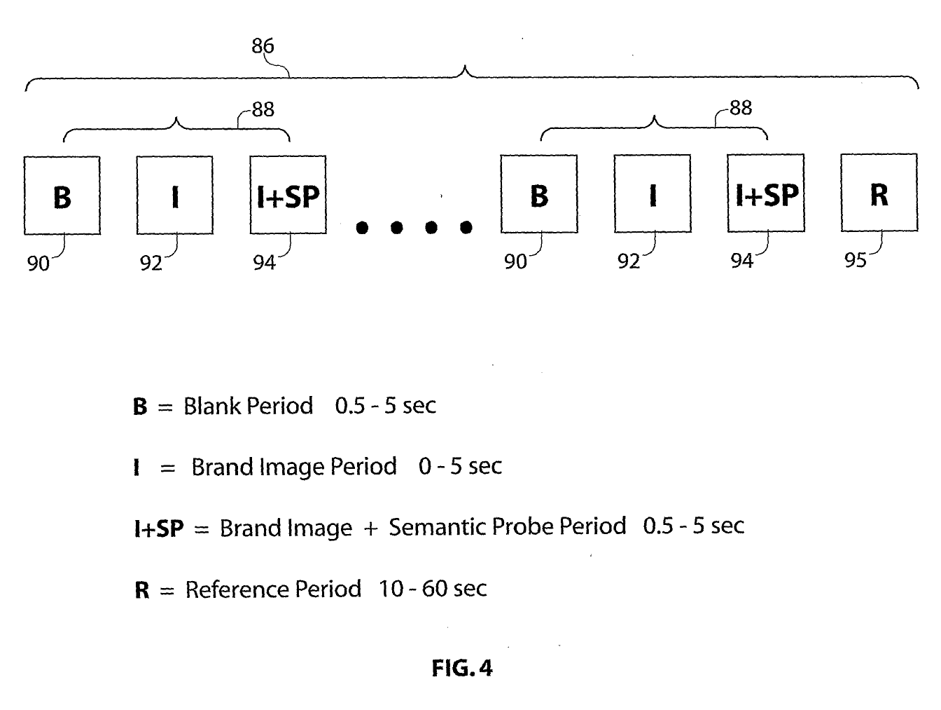

FIG. 4 diagrammatically shows the timing of the presentation of a brand or product image and a semantic probe;

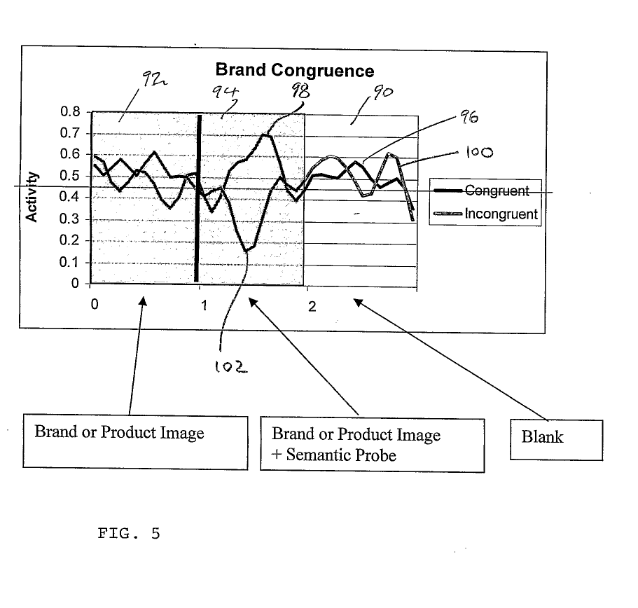

FIG. 5 is a graphical representation of brain activity as a function of time;

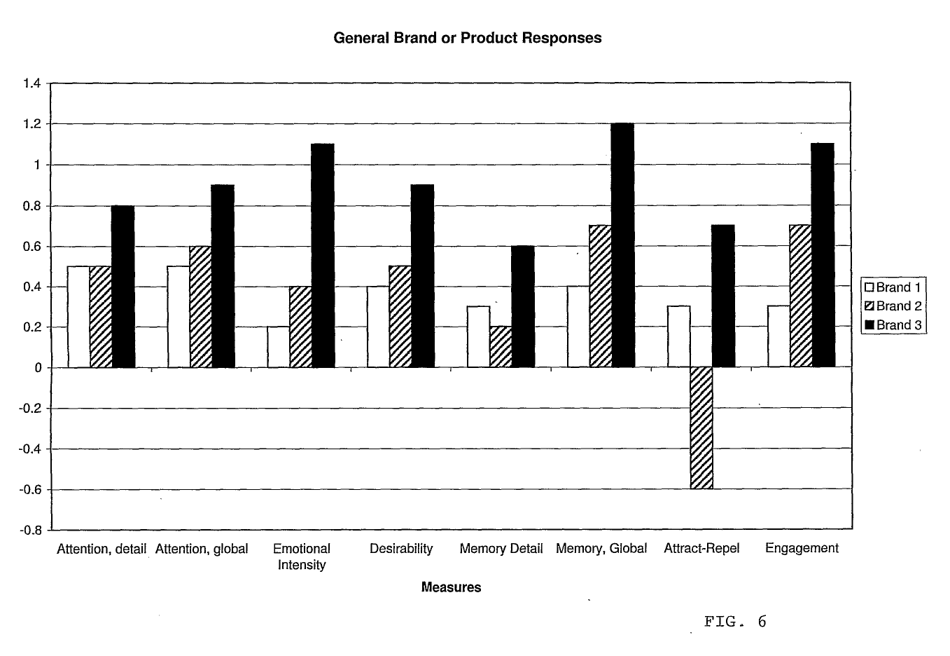

FIG. 6 is a graph showing peak values of brain activity for three different brands; and

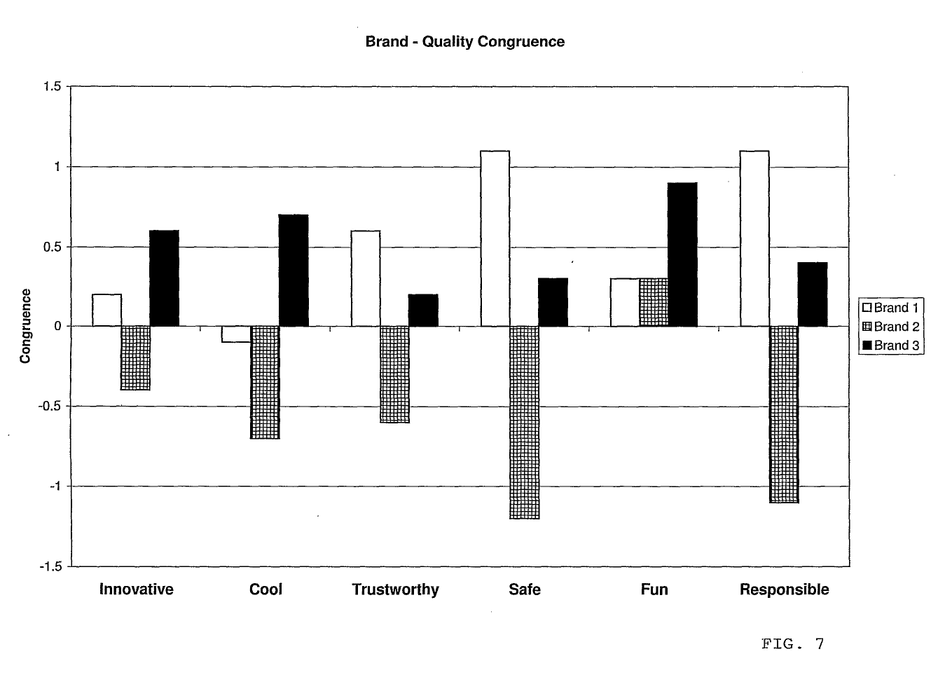

FIG. 7 is a graphical representation illustrating brand congruence associated with three different brands.

FIG. 1 schematically illustrates a system for determining the response of a subject or a group of subjects to audio-visual material presented on a video screen 3 and loudspeaker 2. The system includes a computer 1 which controls various parts of the hardware and also performs computation on signals derived from the brain activity of the subject 7, as will be described below. The computer 1 also holds the images and sounds which can be presented to one or more subjects 7 on the screen 3 and/or through the loudspeaker 2.

The subject or subjects 7 to be tested are fitted with a headset 5 which includes a plurality of electrodes for obtaining brain electrical activity from various sights on the scalp of the subject 7. In the event that the SSVER is used, the recording electrodes in the headset 5 are not used and a commercial MEG recording system such as the CTF MEG System manufactured by VSM MedTech Ltd of 9 Burbidge Street, Coquitlam, BC, Canada, can be used instead. The headset includes a visor 4 which includes half silvered mirrors 8 and white light Light Emitting Diode (LED) arrays 9, as shown in FIG. 2. The half silvered mirrors 8 are arranged to direct light from the LED arrays 9 towards the eyes of the subject 7. The LED arrays 9 are controlled so that the light intensity therefrom varies sinusoidally under the control of control circuitry 6. The control circuitry 6 includes a waveform generator for generating the sinusoidal signal. In the event that the SSVER is used, the light from the LED array 9 is conveyed to the visor via a fibre optic system. The circuitry 6 also includes amplifiers, filters, analogue to digital converters and a USB interface or a TCP interface or other digital interface for coupling the various electrode signals into the computer 1.

A translucent screen 10 is located in front of each LED array 9. Printed on the screen is an opaque pattern. The opacity is a maximum in a circular area in the centre of the screen as shown in FIG. 3. Beyond the circular area, the opacity falls off smoothly with radial distance from the circular area circumference, preferably, the opacity should fall off as a Gaussian function described by Equation 8. The screen reduces the flicker in the central visual field thus giving subjects a clear view of the visually presented material. The size of the central opaque circle should be such as to occlude the visual flicker in the central visual field between 1° to 4° vertically and horizontally.

If r<R then P=1

If r≧R then P is given by Equation 8 below.

P=e^{-(r-R)}^{²}^{/G}^{²} Equation 8
where P is the opacity of the pattern on the translucent screen. An opacity of P=1.0 corresponds to no light being transmitted through the screen while an opacity of P=0 corresponds to complete transparency.

R is the radius of the central opaque disk while r is the radial distance from the centre of the opaque disk. G is a parameter that determines the rate of fall-off of opacity with radial distance. Typically G has values between R/4 and 2R. FIG. 5 illustrates the fall-off of opacity with radial distance from the centre of the disk. In FIG. 5, R=1 and G=2R.

The computer 1 includes software which calculates SSVEP or SSVER amplitude and phase from each of the electrodes in the headset 5 or MEG sensors.

Details of the hardware and software required for generating SSVEP and SSVER are well known and need not be described in detail. In this respect reference is made to the aforementioned United States patent specifications which disclose details of the hardware and techniques for computation of SSVEP. Briefly, the subject 7 views the video screen 3 through the special visor 4 which delivers a continuous background flicker to the peripheral vision. The frequency of the background flicker is typically 13 Hz but may be selected to be between 3 Hz and 50 Hz. More than one flicker frequency can be presented simultaneously. The number of frequencies can vary between 1 and 5. Brain electrical activity will be recorded using specialized electronic hardware that filters and amplifies the signal, digitizes it in the circuit 6 where it is then transferred to the computer 1 for storage and analysis.

When using the SSVEP, brain electrical activity is recorded using multiple electrodes in headset 5 or another commercially available multi-electrode system such as Electro-cap (ECI Inc., Eaton, Ohio USA). When using the SSVER, commercial MEG recording system such as the CTF MEG System manufactured by VSM MedTech Ltd may be used. The number of electrodes or magnetic recording sites is normally not less than 8 and normally not more than 128, typically 16 to 32.

Brain electrical activity at each of the electrodes is conducted to a signal conditioning system and control circuitry 6. The circuitry 6 includes multistage fixed gain amplification, band pass filtering and sample-and-hold circuitry for each channel. Amplified/filtered brain activity is digitized to 16-24 bit accuracy at a rate not less than 300 Hz and transferred to the computer 1 for storage on hard disk. The timing of each brain electrical sample together with the time of presentation of different components of the audio-visual material are also registered and stored to an accuracy 10 microseconds. The equivalent MEG recording system that is commercially available performs the same functions.

While one or more subjects are viewing the images to be evaluated, the visual flicker is switched on in the visor 4 and brain electrical activity is recorded continuously on the computer 1.

At the end of the recording stage, the SSVEP or SSVER amplitude and phase are separately calculated for each individual. Once all recordings are completed, group averaged data is calculated by averaging the smoothed SSVEP or SSVER amplitude and phase data from subjects to be included in the group (e.g. male, female, young, old).

### EXAMPLE 1

The following procedure is used to evaluate the brand attributes for a client. A selected number of subjects, say 50, are seated in a test room and the headsets 5 are placed on their heads. The visors 4 are then placed in position and adjusted so that the foveal block by the screens 10 prevent the appearance of the flicker over the screens 3 where the images are presented. The number of subjects in a recording session is variable and typically can vary from 1 to over 100. When pooling subjects to create the average response, the number of subjects whose data is to be included in the average should be no less than 16.

The brand or product images to be evaluated are presented to the subjects in a particular sequence. FIG. 4 diagrammatically illustrates a typical sequence 86. The sequence itself is made up of a number of blocks 88, each of which commences with a blank period 90, a brand image period 92 in which the brand or product image is displayed on the screen followed by a congruence period 94 in which the same brand or product image of that block 88 and a semantic probe are simultaneously displayed. In the illustrated arrangement, each of the blank periods 90, brand image periods 92 and congruence periods 94 are of the same length which is in the range from 0.5 to 5 secs. The full sequence 86 includes a reference period 95 which follows the last block 88. The reference period has a duration from 10 to 60 secs and preferably about 30 secs in which neutral material such as images of scenery are sequentially displayed. The reference period 95 preferably displays the images of scenery for 0.5 secs and has musical accompaniment.

The sequence 86 may include any convenient number of blocks 88. In a typical evaluation of a brand or product image, there may be three to six blocks 88 presented to the subjects in which different semantic probes are presented during the congruence periods 94. In addition, five to ten different brands may be included in the sequence 86. Accordingly, there may be from fifteen to sixty blocks 88 in the sequence 86.

Brain activity is recorded from the subjects during each of the periods 90, 92 and 94 and reference brain activity is calculated for each subject during the reference period 95.

In a typical assessment, sequences 86 are incorporated into a television program. The first sequence 86 is typically presented early in the television program while the second sequence 86 is presented late in the program, after one or more ‘advertising breaks’ that may be included in the program. It is preferred that the advertising breaks are followed by a similar reference period 95. The reference period 95 preferably displays the images of scenery for 0.5 secs and has musical accompaniment. It is also preferred that the reference periods 95 are the same in the two sequences 86 and are the same after the advertising breaks. Brain activity levels during the reference periods 95 are used as reference levels for brain activity during the preceding blocks 88 and the advertisement breaks. This enables removal of any long-term changes in brain activity that may occur over the time course of the recording period.

Pooled or averaged data at various brain sites can then be displayed to the client as the difference between the reference level and the value at other points in time during the sequence 86. A fixed offset between 0.2 to 0.6 preferably 0.3 radians is then added to the abovementioned difference to yield the SSVEP phase data at each scalp site.

In the sequence illustrated in FIG. 4, each of the blocks 88 commences with a blank period 90. This is thought to be highly preferable so as to properly distinguish brain activity levels between periods 92 and 94 of adjacent blocks 88. It is possible, however, to reduce the duration of the blank periods 90 to zero in which case this could be offset by making the duration of the blocks 92 and 94 much longer so as to enable adequate differentiation between the periods 92 and 94 in adjacent blocks 88.

It is also preferred to have the reference level 95 at the end of the sequence 86. This assists in obtaining a better reference level because if the reference period 95 were at the commencement of the sequence, then the subjects may have some initial interest in whatever material was initially presented and this might lead to somewhat inaccurate results. Where a number of sequences are included in a television program then it is probably less important that the reference period 95 be at the end of the sequences 86 for second and subsequent sequences 86.

To minimize subject irritation or discomfort to the subject due to the flicker, the flicker stimulus is of variable intensity and only switched to the highest intensity when material of interest to the client such as the sequence 86 or advertisement break is present on the screen. During the periods that material of interest is not present on the screen, the stimulus intensity is typically zero and never more than 10% of the typical value used when material of interest is on the screen. Preferably, the stimulus is not switched on abruptly but is slowly increased before each sequence 86 or advertisement break and decreased slowly after the end of each sequence 86 or advertisement break. Typically, the stimulus is increased linearly over a 30-60 second epoch prior to the image block or advertisement break so that it reaches its maximum value 60 seconds prior to the first image sequence or advertisement. Immediately the sequence of reference images of the reference period 95 ends, the stimulus intensity is linearly reduced to the minimum value over a 30 second period. The slow linear increase and decrease of stimulus intensity occurs for every sequence 86 or advertisement break.

### General Brand Characteristics

Once brain activity has been recorded from all subjects, the activity associated with each specific image sequence is averaged across trials for each subject and then across all subjects. Preferably, image sequences presented before and after the advertisement breaks are averaged separately. The General Brand Characteristics or the psychological response to the brand alone is indicated by the peak value of the brain activity at the above listed scalp sites during the period that the brand or product image is presented alone. More specifically, peak brain activity is assessed during brand image period 92 of FIG. 4, from which is subtracted brain activity assessed during the reference period 95. The psychological responses to the brand or product image thus include:
the level of attention to detail elicited by the brand or product image;
the level of attention to global features elicited by the brand or product image;
the level of desirability elicited by the brand or product image;
the level of emotional intensity elicited by the brand or product image;
the level of memory encoding for text and detail elicited by the brand or product image;
the level of memory encoding for emotions or imagery elicited by the brand or product image;
the extent to which the brand or product image elicits feelings of attraction or repulsion; and
the extent to which the brand or product image engages subjects.

These responses are determined as described above.

FIG. 6 illustrates the peak value of the above measures for three hypothetical brands, Brand 1, Brand 2 and Brand 3. In this example, Brand 1 is a frozen vegetable product brand, Brand 2 a tobacco product brand while Brand 3 is a global airline and mobile phone brand. While Brand 1 elicits low to moderate levels of the various measures (as labelled in FIG. 6), Brand 2 elicits a higher level of emotional intensity, global memory encoding and engagement and a strong repulsion. Brand 3 elicits the strongest levels of attention, emotional intensity, emotional memory, engagement and a strong attraction. This data would inform corporate brand managers that Brand 1 has a relatively weak brand presence that is generally neutral to positive. Brand 2, on the other hand elicits stronger emotional intensity and engagement indicating a strong emotional presence. However, subjects are repelled by the brand indicating an active dislike of the brand. By contrast, Brand 3 elicits very high levels of attention, emotional intensity, engagement and strong attraction. This brand has a very high presence that is very positive. These data would indicate that the subject group considers Brand 1 of little personal relevance and a weak motivator for brand loyalty. Brand 2 is negatively perceived and the subject group would actively avoid this brand. Brand 3 has a very strong and positive brand presence that is consistent with subjects having feelings of high brand loyalty to Brand 3.

The General Brand Characteristics can be measured a number of times to examine the change in these Brand Characteristics. The impact of an advertisement or the television program can be assessed by determining the change in Brand Characteristics or Brand Characteristics after viewing television program or advertisement or program minus Brand Characteristics before viewing television program or advertisement.

Long term changes in brand perception can also be assessed by measuring Brand Characteristics repeatedly over a period of time. These are termed Brand Characteristic tracking studies and the period between measurements can vary from weeks (for advertisement tracking) to months (for brand tracking).

### Specific Brand Characteristics

### EXAMPLE 2

The congruence between ideas and feelings associated with a brand and a specific quality, or Specific Brand Characteristics can be determined from the brain responses elicited by the simultaneous appearance of the brand or product image and the semantic probe. Specific brand characteristics can be determined by reference to differences between the reference level of activity during the reference period 95 and brain activity when viewing the Image-semantic probe combinations during the congruence periods 94. In this Example, 50 subjects viewed twenty corporate logos (representing brands) in the periods 92 and each logo was presented twice followed by congruence periods 94 in which one of the congruence periods included a semantic probe which was generally consistent with the perception of the brand followed by congruence periods in which the semantic probe was generally inconsistent with the perception of the brand. Responses to congruent and incongruent combinations were averaged separately across trials and individuals. While congruent combinations elicited an increase or positive measure of activity at this site, incongruent combinations gave rise to a reduction or a negative measure of activity.

The congruence between the brand or product image and the semantic probe is indicated by the peak value of brain activity at the right prefrontal site located in the vicinity of electrode Fₚ₂ in the International 10-20 system. If inverse mapping techniques are used, the relevant cortical location is the right orbitofrontal cortex in the vicinity of Brodman area 11.

FIG. 5 illustrates brand congruence as determined for one of the twenty corporate logos which were included in the sequence 86. Similar graphical results could be produced for the other nineteen corporate logos but it is unnecessary to describe all of these in detail. More particularly, FIG. 5 shows the period 92 in which the brand or product image is shown followed by the congruence period 94 in which the brand or product image and semantic probe are shown followed by the blank period 90. In this case each of the periods 92, 84 and 90 is of 1 second duration. The line 96 indicates congruence between the semantic probe and the subjects' perception of the brand or product image. It will be seen that the line 96 includes a peak 98 which indicates strong consistency between the semantic probe and the subject's perception of the brand or product image.

FIG. 5 also shows a line 100 which illustrates incongruence between the brand or product image and the semantic probe. For instance, if the product were an automobile noted for safety, the semantic probe could be the word “unsafe” and this generates a trough 102 indicating incongruence between the subjects' perception of the brand or product image and the semantic probe. The ability to measure incongruence is a useful tool for clients to assess the perception of brands or product images against various adverse characteristics, as indicated by the semantic probe.

### EXAMPLE 3

This Example is similar to Example 2 except that three hypothetical brands were included in the brand periods 90 and six different semantic probes were included in the congruence periods 94. Brain activities were recorded during each of the periods 90, 92 and 94 as well as the reference periods 95. FIG. 7 illustrates graphically the congruence measure between the six semantic probes, “Innovative”, “Cool”, “Trustworthy”, “Safe”, “Fun” and “Responsible” and the three hypothetical brands (Brand 1, a vehicle brand known for its emphasis on safety, Brand 2 a cigarette brand and Brand 3, an airline and mobile phone brand. The graphical results indicate that Brand 1 is viewed by the subjects as trustworthy, safe and responsible, while lacking in fashion or fun as indicated by negative or low levels to the semantic probes “cool” and “fun”. Brand 2 is viewed very negatively as unfashionable, unsafe and untrustworthy as indicated by strongly negative assessments to all the semantic probes except the word “fun” which is low positive. Brand 3 is viewed as fashionable and fun as indicated by high positive responses to the semantic probes “cool” and “fun”.

These measures offer brand managers an objective and more accurate indication of the way a brand is perceived and also changes in brand perception. Such changes may be a result of actions taken by the company such as advertising or sponsorship or may be a consequence of desired or undesired publicity. Any undesired changes in brand perception can be detected early and appropriate action taken.

Many modifications will be apparent to those skilled in the art without departing from the spirit and scope of the invention.

1. A method of evaluating characteristics of a brand or product including the steps of:
(a) presenting the brand or product to the subject during a first period;
(b) determining brain activity of the subject during the first period;
(c) presenting neutral visual and/or audio material to a subject during a second period;
(d) determining a reference level of brain activity of the subject during the second period; and
(e) evaluating attributes associated by the subject with the brand or product by determining differences in brain activities between said first and second periods.

2. A method as claimed in claim 1 including the step of displaying steps (a) and (c) as a presentation sequence.

3. A method as claimed in claim 2 wherein there are a plurality of steps (a) in each presentation sequence and wherein blank periods are presented between successive steps (a).

4. A method as claimed in claim 3 wherein the first periods have a duration of 0.5 to 5 seconds, the second periods have a duration of 10 to 60 seconds and the blank periods have a duration of 0 to 5 seconds.

5. A method as claimed in claim 3 wherein the second period is at the end of said sequence.

6. A method as claimed in claim 1 wherein steps (a) to (d) are presented to a plurality of subjects and step (e) includes the steps of averaging the differences in brain activities of the subjects.

7. A method as claimed in claim 1 wherein step (a) is carried out by displaying the brand or product on a video screen.

8. A method as claimed in claim 1 wherein steps (b) and (d) are carried out by determining gamma or high frequency EEG or MEG activity.

9. A method as claimed in claim 1 wherein steps (b) and (d) are carried out by detecting EEG or MEG activity in the frequency range 8 to 13 Hz.

10. A method as claimed in claim 1 wherein steps (b) and (d) are carried out by assessment of the phase of steady state visually evoked potentials (SSVEP) in EEG signals obtained from the subject or subjects or by assessment of steady state visually evoked responses (SSVER) in MEG signals obtained from the subject or subjects.

11. A method as claimed in claim 1 wherein steps (a) and (c) include the steps of placing electrodes at scalp sites to obtain output EEG signals which enable assessment of:
visual attention to detail of the brand or product;
visual attention to global features of the brand or product;
desirability of the brand or product;
emotional intensity associated with the brand or product;
long term memory encoding associated with the brand or product;
engagement with the brand or product; and/or attraction associated with the brand or product.

12. A method as claimed in claim 11 including the step of applying a sinusoidally varying visual flicker stimulus to each subject during steps (a) and (c) to thereby enable calculation of Fourier coefficients from said output signals to thereby enable calculation of said SSVEP amplitudes and/or phase differences.

13. A method as claimed in claim 12 wherein said SSVEP amplitude and phase are calculated by the equations:
$${SSVEP}_{amplitude} = \sqrt{( {A}_{n}^{2} + {B}_{n}^{2} )}$$
$${SSVEP}_{phase} = atan ( \frac{{B}_{n}}{{A}_{n}} )$$
where: aₙ and bₙ are cosine and sine Fourier coefficients calculated by the equations:
$${a}_{n} = \frac{1}{S Δτ} {\sum }_{i = 0}^{S - 1} f ( nT + i Δτ ) cos ( \frac{2 π}{T} ( nT + i Δτ ) )$$
$${b}_{n} = \frac{1}{S Δτ} {\sum }_{i = 0}^{S - 1} f ( nT + i Δτ ) sin ( \frac{2 π}{T} ( nT + i Δτ ) )$$
where:
aₙ and bₙ are the cosine and sine Fourier coefficients respectively where;
n represents the nth flicker stimulus cycle;
S is the number of samples per flicker stimulus cycle;
Δτ is the time interval between samples;
T is the period of one cycle;
f(nT+iΔτ) is the EEG signal (raw or pre-processed using ICA) obtained from said predetermined scalp sites;
and wherein Aₙ and Bₙ are overlapping smoothed Fourier coefficients calculated by using the equation:
$${A}_{n} = {\sum }_{i = 1}^{i = N} {a}_{n + i} / N$$
$${B}_{n} = {\sum }_{i = 1}^{i = N} {b}_{n + i} / N$$

14. A method as claimed in claim 13 including the steps of:
obtaining EEG signals from a plurality of scalp sites of each subject; and
utilising inverse mapping techniques such as BESA, EMSA or LORETA to produce modified EEG signals which represent activity in deeper regions of the brain of each subject such as the orbito-frontal cortex or the ventro-medial cortex.

15. A method as claimed in claim 13 including the step of averaging the Fourier coefficients Aₙ and Bₙ for a selected group of subjects and then calculating the SSVEP amplitudes and SSVEP phase differences for said group of subjects.

16. A method as claimed in claim 12 wherein the flicker signal is applied only to the peripheral vision of each subject.

17. A method as claimed in claim 16 including the steps of directing the light towards the eyes of each subject via first and second screens so that the light passing through the screen constitutes said flicker signal and wherein each screen includes an opaque area, and wherein the method further includes the step of positioning the screens to the relative position of each subject such that said opaque areas prevent said flicker signal impinging on the fovea of each eye of each subject.

18. A method as claimed in claim 17 wherein the opacity of each screen decreases as a function of distance from its opaque area so that the intensity of the flicker signal impinging on each retina of each subject decreases in value from the central vision to the peripheral vision.

19. A method as claimed in claim 18 including the step of applying a masking pattern to each screen to define the opacity thereof, the method including the step of applying the pattern in accordance with a masking pattern function which provides zero or low gradients for changes in opacity adjacent to its opaque area and peripheral areas thereof which define parts of the flicker signal impinging on the peripheral vision of each subject.

20. A method as claimed in claim 19 wherein the opaque area of each screen is circular and wherein the masking pattern function is selected to be a Gaussian function, so that the opacity P of the screen is defined by the equation:
P=e^{-(r-R)}^{²}^{/G}^{²}
where:
r is the radial distance from the centre of the opaque area; and
G is a parameter that determines the rate of fall-off of opacity with radial distance, and wherein when r<R, P=1.

21. A method as claimed in claim 20 wherein G has a value in the range R/4 and 2R.

22. A method as claimed in claim 13 including the steps of applying an electrode to the scalp of each subject at the O₁ site, calculating SSVEP amplitudes and phase differences from EEG signals from said electrode whereby the output signals indicate each subject's visual attention to details of the brand or product.

23. A method as claimed in claim 14 including the step of utilising inverse mapping determines brain activity in the left cerebral cortex in the vicinity of Brodman's area 17 whereby the modified output signals indicate each subject's visual attention to details of the brand or product.

24. A method as claimed in claim 13 including the steps of applying an electrode to the scalp of each subject at the O₂ site, calculating SSVEP amplitudes and phase differences from EEG signals from said electrode whereby the output signals are indicative of each subject's visual attention to global features of the brand or product.

25. A method as claimed in claim 14 including the step of utilising inverse mapping determines brain activity in the right cerebral cortex in the vicinity of Brodman's area 17 whereby the output signals indicate each subject's visual attention to global features of the brand or product.

26. A method as claimed in claim 13 including the step of applying an electrode to the scalp of each subject at the P₃ site, calculating SSVEP amplitudes and phase differences from EEG signals from said electrode whereby the output signals are indicative of each subject's multi-modal attention to detail or desirability to features of the brand or product.

27. A method as claimed in claim 14 wherein the step of utilising inverse mapping determines brain activity in the left cerebral cortex in the vicinity of the intraparietal area whereby the output signals indicate each subject's multi-modal attention to detail or desirability of the features of the brand or product.

28. A method as claimed in claim 13 including the step of applying an electrode to the scalp of each subject at the P₄ site, calculating SSVEP amplitudes and phase differences from EEG signals from said electrode whereby the output signals indicate each subject's multi-modal attention to global features or desirability of the features of the brand or product.

29. A method as claimed in claim 14 wherein the step of utilising inverse mapping determines brain activity in the right cerebral cortex in the vicinity of the intraparietal area whereby the output signals indicate each subject's multi-modal attention to global features or desirability of the features of the brand or product.

30. A method as claimed in claim 13 including the step of applying an electrode to the scalp of each subject at a site which is approximately equidistant from sites O₂, P₄ and T₆, calculating SSVEP amplitudes and phase differences from EEG signals from said electrode whereby the output signals indicate each subject's emotional intensity associated with the brand or product.

31. A method as claimed in claim 14 wherein the step of utilising inverse mapping determines brain activity in the right cerebral cortex in the vicinity of the right parieto-temporal junction whereby the output signals indicate each subject's emotional intensity associated with the brand or product.

32. A method as claimed in claim 13 including the steps of applying an electrode to the scalp of each subject at the F₃, F₄, Fₚ₁ and Fₚ₂ sites, calculating SSVEP amplitudes and phase differences from EEG signals from said electrodes, calculating values for attraction-repulsion using the equation:
attraction=(a₁*SSVEP phase advance at electrode F₃+a₂*SSVEP phase advance at electrode Fₚ₁−a₃*SSVEP phase advance at electrode F₄−a₄*SSVEP phase advance at electrode Fₚ₂)
where a₁=a₂=a₃=a₄=1.0
whereby said values indicate each subject's attraction or repulsion towards features of the brand or product.

33. A method as claimed in claim 14 wherein the step of utilising inverse mapping determines brain activity in:
the right orbito-frontal cortex in the vicinity of Brodman area 11;
the right dorso-lateral prefrontal cortex in the vicinity of Brodman area 9;
the left orbito frontal cortex in the vicinity of Brodman area 11; and
the left dorso-lateral prefrontal cortex in the vicinity of Brodman area 9; and
calculating a value for attraction-repulsion using the equation:
attraction=(c₁*right orbito-frontal cortex (in vicinity of Brodman area 11)+c₂*right dorso-lateral prefrontal cortex (in vicinity of Brodman area 9)+c₃*left orbito frontal cortex (in vicinity of Brodman area 11)+c₄*left dorso-lateral prefrontal cortex (vicinity of Brodman area 9))
where c₁=1, c₂=1, c₃=1, c₄=1,
whereby said values indicate each subject's attraction or repulsion towards features of the brand or product.

34. A method as claimed in claim 13 including the steps of applying electrodes to the scalp of each subject at F₃, F₄, Pₚ₁ and Fₚ₂ sites, calculating SSVEP amplitudes and phase differences from said electrodes, calculating values for engagement in features of the advertisement by a weighted mean SSVEP phase advance at said sites using the equation:
engagement=(b₁*SSVEP phase advance at electrode F₃+b₂*SSVEP phase advance at electrode Pₚ₁+b₃*SSVEP phase advance at electrode F₄+b₄* SSVEP phase advance at electrode Fₚ₂)
where b₁=0.1, b₂=0.4, b₃=0.1, b₄=0.4,
whereby said values indicate each subject's engagement in features of the brand or product.

35. A method as claimed in claim 14 wherein the step of utilising inverse mapping determines brain activity in:
the right orbito frontal cortex in the vicinity of Brodman area 11;
the right dorso-lateral prefrontal cortex in the vicinity of Brodman area 9;
the left frontal cortex in the vicinity of Brodman area 11; and
the left dorso-lateral prefrontal cortex in the vicinity of Brodman area 9,
calculating SSVEP amplitudes and phase differences from said modified EEG signals from said electrodes; and
calculating a value for engagement using the equation:
engagement=(d₁*right orbito frontal cortex (in vicinity of Brodman area 11)+d₂*right dorso-lateral prefrontal cortex (in vicinity of Brodman area 9)+d₃*left orbito frontal cortex (in vicinity of Brodman area 11)+d₄*left dorso-lateral prefrontal cortex (in vicinity of Brodman area 9))
where d₁=0.1, d₂=0.4, d₃=0.1, d₄=0.4,
whereby said values indicate each subject's engagement in features of the brand or product.

36. A method as claimed in claim 1 including the steps of:
(f) simultaneously presenting the brand or product and a semantic probe to the subject during a third period;
(g) determining brain activity of the subject during the third period; and
(h) determining whether there is congruence or incongruence between the attributes associated with the brand or product and the semantic probe by assessing whether there is an increase or decrease in brain activity in step (g) compared to step (d).

37. A method of determining attributes associated with a brand or product including the steps of:
(a) simultaneously presenting the brand or product and a semantic probe to the subject during a first period;
(b) determining brain activity of the subject during the first period;
(c) presenting the neutral visual and/or audio material to a subject during a second period;
(d) determining a reference level of brain activity of the subject during said second period; and
(e) determining whether there is congruence or incongruence between the attributes associated with the brand or product and the semantic probe by assessing whether there is an increase or decrease in brain activity in step (b) compared to step (d).

38. A method as claimed in claim 37 including applying one or more electrodes to the scalp of the subject such that steps (b) and (d) determine brain activities at the right prefrontal site.

39. A method as claimed in claim 37 including the step of displaying steps (a) and (c) as a presentation sequence.

40. A method as claimed in claim 39 wherein each presentation sequence includes a plurality of presentation blocks and ends with step (c) and wherein each presentation block includes step (a).

41. A method as claimed in claim 40 wherein each presentation block includes a third period in which the brand or product is presented to the subject.

42. A method as claimed in claim 39 wherein each presentation block includes a blank period at the beginning or end thereof during which no visual and/or audio material is presented to the subject.

43. A method as claimed in claim 37 wherein steps (a) to (d) are presented to a plurality of subjects and step (e) includes the steps of averaging the differences in brain activities of the subjects.

44. A method as claimed in claim 37 wherein the first period has a duration in the range 0.5 to 5 seconds.

45. A method as claimed in claim 37 wherein the second period has a duration in the range 10 to 60 seconds.

46. A method as claimed in claim 42 wherein the third period has a duration in the range 0.5 to 5 seconds.

47. A method as claimed in claim 42 wherein the blank period has a duration in the range 0 to 5 seconds.

48. A method as claimed in claim 47 wherein the first, third and blank periods are of equal length.

49. A method as claimed in claim 37 wherein the semantic probe is a word or words.

50. A method as claimed in claim 49 including the step of displaying the semantic probe as text.

51. A method as claimed in claim 50 wherein step (a) is carried out by displaying the brand or product on a video screen and the text also displayed on the video screen.

52. A method as claimed in claim 37 wherein steps (b) and (d) are carried out by determining gamma or high frequency EEG or MEG activity.

53. A method as claimed in claim 37 wherein steps (b) and (d) are carried out by detecting EEG or MEG activity in the frequency range 8 to 13 Hz.

54. A method as claimed in claim 37 wherein steps (b) and (d) are carried out by assessment of the phase of steady state visually evoked potentials (SSVEP) in EEG signals obtained from the subject or subjects or by assessment of steady state visually evoked responses (SSVER) in MEG signals obtained from the subject or subjects.

55. A method as claimed in claim 37 wherein steps (b) and (d) are carried out by assessment of EEG signals from the Fps electrode of the subject or each subject.

56. A method as claimed in claim 37 wherein steps (b) and (d) are carried out by assessment of EEG signals and including the step of applying inverse mapping techniques to determine brain activity at the right orbito-frontal cortex in the vicinity of Brodman area 11.

57. A system for determining attributes associated with a brand or product including:
display means for displaying the brand or product image to a subject;
brain activity determining means for determining brain activity of the subject; and
assessment means coupled to receive first output signals from said brain activity determining means in a first period in which the brand or product image is displayed to the subject and to receive second output originals from said brain activity determining means in a second period in which neutral material is displayed to the subject in order to establish a reference level of brain activity, the assessment means being operable to assess differences between said first and second output signals.

58. A system for determining attributes associated with a brand or product including:
display means for displaying the brand or product image to a subject;
brain activity determining means for determining brain activity of the subject; and
assessment means coupled to receive first output signals from said brain activity determining means in a first period in which the brand or product image is displayed to the subject simultaneously with a semantic probe and to receive second output originals from said brain activity determining means in a second period in which neutral material is displayed to the subject in order to establish a reference level of brain activity, the assessment means being operable to assess differences between said first and second output signals.
